(19) United States
(12) Patent Application Publication
(10) Pub. No.: US 2010/0160440 A1
(43) Pub. Date: Jun. 24, 2010

(54) INTRANASAL PHARMACEUTICAL COMPOSITIONS COMPRISING SUCCINIC ACID AND METHODS THEREOF

(76) Inventor: Igor Anatolievich Pomytkin, Moscow (RU)

Correspondence Address: NOTARO, MICHALOS & ZACCARIA P.C., 100 DUTCH HILL ROAD, ORANGEBURG, NY, 10962, US

(21) Appl. No.: 12/670,690

(22) PCT Filed: Aug. 2, 2007
(86) PCT No.: PCT/RU07/00421

§ 371 Date: Feb. 3, 2010

(51) Int. Cl. A61K 31/194

(52) U.S. Cl. 514/574

(57) ABSTRACT

The present invention relates to intranasal pharmaceutical compositions for preventing and/or treating a disease selected from the group consisting of Alzheimer's disease, Parkinson's disease, cerebral ischemia and neurological damage due to stroke comprising a therapeutically effective amount of succinic acid or a pharmaceutically acceptable salt thereof, and a pharmaceutically and intranasally acceptable carrier. Further, the present invention relates to methods for preventing and/or treating a disease selected from the group consisting of Alzheimer's disease, Parkinson's disease, cerebral ischemia and neurological damage due to stroke with using compositions of the present invention.

### FIELD OF THE INVENTION

The invention relates to intranasal pharmaceutical compositions comprising succinic acid or pharmaceutically acceptable salts thereof for preventing and/or treating neurodegenerative diseases.

### BACKGROUND OF THE INVENTION

There is grown evidence that insulin acts in brain to regulate a variety of brain functions including learning and memory. Neurodegenerative diseases such as Alzheimer's disease and Parkinson's disease are accompanied with central insulin resistance. Craft S, Neurobiol Aging 2005, 26(Suppl 1):65-9. Hoyer S, Eur J Pharmacol 2004, 490(1-3):115-25. Steen E et al., J Alzheimers Dis 2005, 7:63-80. Moroo I et al., Acta Neuropathol (Berl). 1994;87(4):343-8. Thus, there is the need to improve central insulin sensitivity to treat neurodegenerative diseases.

U.S. Pat. No. 6,521,665 discloses the method of treating insulin resistance comprising administering to a mammal in need thereof an effective amount of succinic acid or a pharmaceutically acceptable salt thereof, wherein the succinic acid or a pharmaceutically acceptable salt thereof is administered orally, or parenterally, or topically, or rectally. However, succinic acid does not manifest central effects under routes of administration disclosed in U.S. Pat. No. 6,521,665, because of a very low transport capacity for four-carbon dicarboxylates across the blood-brain barrier (BBB). Hassel B et al, J Neurochem 2002; 82(2):410-9.

Surprisingly, it is demonstrated in the present invention that succinic acid manifests central effects under intranasal administration.

It is an object of the present invention to provide an intranasal pharmaceutical composition for preventing and/or treating neurodegenerative diseases comprising a therapeutically effective amount of succinic acid or a pharmaceutically acceptable salt thereof.

It is an object of the present invention to provide a method for treating neurodegenerative diseases comprising intranasally administering a pharmaceutical composition comprising a therapeutically effective amount of succinic acid or a pharmaceutically acceptable salt thereof.

### DETAILED DESCRIPTION OF THE INVENTION

The present invention provides an intranasal pharmaceutical composition for preventing and/or treating a disease selected from the group consisting of Alzheimer's disease, Parkinson's disease, cerebral ischemia and neurological damage due to stroke comprising a therapeutically effective amount of succinic acid or a pharmaceutically acceptable salt thereof and a pharmaceutically and intranasally acceptable carrier.

The term <<pharmaceutically acceptable salt” refers to non-toxic base addition salts. The pharmaceutically acceptable salts of the invention are prepared by a reaction of succinic acid with a pharmaceutically acceptable base by methods well-known from the art. Such bases include, but are not limited to, ammonia; sodium base; potassium base; organic amines like as triethylamine, ethanolamine, dimethylethanolamine, diethanolamine, and triethanolamine; 2-ethyl-6-methyl-3-hydroxypiridine; and basic amino acids like arginine, ornithine, and lysine.

The term “therapeutically effective amount” refers to a nontoxic but sufficient amount of succinic acid or a pharmaceutically acceptable salt thereof to provide the desired therapeutic effect. Preferably, the therapeutically effective amount of succinic acid or a pharmaceutically acceptable salt thereof is from 0.01 to 30 mg per a unit dosage form of the composition of the present invention, more preferably, from 5 to 15 mg per the unit dosage form.

The term “intranasal administration” refers to delivery of the composition to any portion of the nasal epithelium.

The term <<pharmaceutically and intranasally acceptable carrier” refers to a one or more compatible solid or liquid filler diluents or encapsulating substances which are suitable for administration to any portion of the nasal epithelium of a mammal, preferably a human. Typically, the carrier may be a liquid, solution, suspension, gel, ointment, lotion, or combinations thereof. Preferably, the carrier is a pharmaceutically acceptable aqueous carrier.

The compositions of the invention are prepared by methods well-known from the art in accordance with accepted pharmaceutical procedures, for example, as described in Remington's Pharmaceutical Sciences, seventeenth edition, ed. Alfonso R. Gennaro, Mack Publishing Company, Easton, Pa., Eighteenth edition (1990).

The compositions of the invention can be prepared in a variety of unit dosage forms. Such forms are include, but are not limited to, nasal drop, nasal spray, nasal gel, nasal ointment, and nasal powder. The content of succinic acid or a pharmaceutically acceptable salt thereof should be in the range from 0.1 to 90%, preferably 0.5 to 10% by the weight of the composition.

Further, the present invention provides a method for preventing and/or treating a disease selected from the group consisting of Alzheimer's disease, Parkinson's disease, cerebral ischemia and neurological damage due to stroke comprising intranasally administering to a mammal in need thereof a pharmaceutical composition comprising a therapeutically effective amount of succinic acid or a pharmaceutically acceptable salt thereof, and a pharmaceutically and intranasally acceptable carrier.

As used herein, the term “treating a disease” means treating, controlling, preventing and/or reducing one or more clinical signs (i.e., symptoms) of the disease in a mammal in need thereof.

Preferably, the therapeutically effective amount in the method of the invention is 0.01 to 5 mg per kilogram of body weight of the mammal, more preferably, 0.1 to 1 mg per kilogram.

Nonexclusive examples of mammals of the invention include humans and companion animals such as cats and dogs. Preferably, the mammal is a human.

The following examples are presented to demonstrate the invention. The examples are illustrative only and are not intended to limit the scope of the invention in any way.

### EXAMPLE 1

This example demonstrates compositions for intranasal administration comprising succinic acid or a pharmaceutically acceptable salt thereof.

| Ingredient | Content |
|---|---|
| Succinic acid | 50 mg/ml |
| Disodium phosphate USP/Ph Eur | qs to pH 5.0 |
| Water for injections USP/Ph Eur | to 1.0 ml |

Succinic acid is dissolved in water for injection to the desired volume, 0.4M disodium phosphate is added to pH 5.0. In this manner, solution with concentration of succinic acid of 50 mg/ml is prepared. The solution is filtered through a sterilizing grade filter (0.2 μm), and filled into USP/Ph Eur Type 1 glass vials (nominal spray volume 100 μL) which are closed with chlorobutyl stoppers. The vials are assembled into the commercially available unit dose nasal spray device. The assembled device may be used to deliver unit doses of succinic acid of 5.0 mg in a single administration. The filled nasal spray device is packaged into a plastic tray and placed inside a carton to provide protection from the light.

The patient removes the packaging from the nasal spray device which contains a sterile solution of succinic acid and then inserts the nozzle of the device into a nostril and articles is to administer a single dose.

### EXAMPLE 2

This example demonstrates methods for treating neurodegenerative disorders in mammals in need thereof.

A disease relevant to human Alzheimer's disease was induced by injection of beta-amyloid peptide 25-35 (beta-amyloid) into nucleus basalis magnocellularis (NBM) of rat brains as described by Harkany T et al. in Behav Brain Res. 1998 90(2):133-45. Beta-amyloid was administered bilaterally into NBM of male Wistar rats in dose of 2 μg per each side. On day 16^{th} after the amyloid injection, rats received intranasally or intraperitoneally compositions comprising a water solution of 1 mg/kg of succinic acid for 7 days singly a day. Control rats received saline intranasally. On day next to the last day of the treatment, passive avoidance performance in rats was tested for two consecutive days. A two-compartment, step-through, passive avoidance apparatus consisting of illuminated (25×40×25 cm) and dark (25×40×25 cm) compartments attached to an electrified grid floor and separated by a guillotine door (8×8 cm) was used. In the acquisition trial, the rat was placed in the illuminated compartment in a position its tail directed to the closed door for 2 min to habituate to the apparatus. The guillotine door was opened and time to enter to dark compartment was recorded. When the rat entered to dark compartment completely (four foots in dark compartment), the guillotine door was closed and the rat was delivered an electrical shock of 0.8 mA for 3 sec through the grid floor. After the shock, the rat was immediately placed in home cage. In the retention trial, conducted 24 h after the acquisition trial, the rat was placed in the illuminated compartment and the retention latency to enter into the dark compartment was recorded until 180 s had elapsed. The latency was accepted for 180 s, if the rat did not enter the dark compartment for 180 s. Data are presented as retention latency mean±SD (n=8).

| Groups | Latency, s |
|---|---|
| Control | 48 ± 11 |
| Succinic acid, intranasally | 87 ± 21* |
| Succinic acid, intraperitoneally | 43 ± 19 |

*Differs significantly of control (P < 0.05).

Thus, intranasal administration of composition comprising succinic acid is much more effective than intraperitoneal administration. Intranasally treated rats demonstrate significant improvement in learning and memory as compared to control rats, whereas intraperitoneally treated rats do not.

What is claimed is:

1. An intranasal pharmaceutical composition for preventing and/or treating a disease selected from the group consisting of Alzheimer's disease, Parkinson's disease, cerebral ischemia and neurological damage due to stroke comprising a therapeutically effective amount of succinic acid or a pharmaceutically acceptable salt thereof, and a pharmaceutically and intranasally acceptable carrier.

2. A method for preventing and/or treating a disease selected from the group consisting of Alzheimer's disease, Parkinson's disease, cerebral ischemia and neurological damage due to stroke comprising intranasally administering to a mammal in need thereof a pharmaceutical composition comprising a therapeutically effective amount of succinic acid or a pharmaceutically acceptable salt thereof, and a pharmaceutically and intranasally acceptable carrier.
